(19) United States
(12) Patent Application Publication
(10) Pub. No.: US 2025/0345088 A1
(43) Pub. Date: Nov. 13, 2025

(54) ATHERECTOMY SYSTEM AND METHOD FOR USING SAME

(71) Applicant: ASAHI INTECC CO., LTD., Seto-shi (JP)

(72) Inventor: Hirobumi Watanabe, Irvine, CA (US)

(21) Appl. No.: 19/199,636

(22) Filed: May 6, 2025

Related U.S. Application Data
(60) Provisional application No. 63/643,598, filed on May 7, 2024.

(51) Int. Cl. A61B 17/3207; A61B 17/00; A61B 17/32; A61B 90/00

(52) U.S. Cl. CPC A61B 17/320758; A61B 2017/00561; A61B 2017/320052; A61B 2090/3966

(57) ABSTRACT

Embodiments of the present disclosure relate to an atherectomy system and method for using same. An example atherectomy system includes at least one wire comprising opposed ends, the opposed ends defining a length of the at least one wire; a helical screw coupled to the at least one wire along the length, wherein the helical screw is configured to rotate with the at least one wire; and a tubular element threaded over a portion of the length. The tubular element may comprise a tip comprising at least one blade structure. The helical screw may be configured to, upon rotation of the at least one wire, generate a negative pressure within the tubular element. The negative pressure within the tubular element may be configured to draw material toward the tip to enable cutting of the material against the helical screw and the at least one blade structure.

### CROSS REFERENCE TO RELATED APPLICATIONS

This application claims the benefit of and priority to U.S. Application No. 63/643,598, filed May 7, 2024, entitled “ATHERECTOMY SYSTEM AND METHOD FOR USING SAME,” the disclosure of which is incorporated herein by reference in its entirety.

### TECHNICAL FIELD

This application generally relates to systems and methods for disrupting and removing material from a tubular structure.

### BACKGROUND

An atherectomy procedure may be performed to remove atherosclerotic plaque buildup from arteries. For example, a subject suffering from atherosclerosis may present with arterial wall buildups of fatty deposits, cholesterol, calcium, and other substances, which risk narrowing and hardening the artery. Typical approaches to performing an atherectomy utilize a catheter device comprising a cutting, grinding, or light emissive element for ablating material within an artery. For example, such devices may be inserted to a target site within an artery and activated to break up plaque deposits, which may be subsequently extracted via suction or flushing. However, existing approaches may be unsuitable for certain blood vessels due to complexities of miniaturizing the devices, increased risk of complications, and reductions in plaque debulking efficiency and output. For example, small target sites may increase a risk that bladed mechanisms, grinding mechanisms, and/or the like contact and damage blood vessel walls. As another example, devices that rotate along an eccentric motion path may fail to achieve complete rotation within small-diameter blood vessels, and, instead, may collide with blood vessel walls. Thus, existing approaches have yet to solve the challenge of minimizing atherectomy devices for target sites of small diameter.

### BRIEF SUMMARY

Embodiments of the present disclosure relate to atherectomy systems, atherectomy kits, and methods for using the same. An example atherectomy system of the present disclosure may include a at least one wire comprising opposed ends, the opposed ends defining a length of the at least one wire; a helical screw coupled to the at least one wire along the length of the at least one wire between the opposed ends, wherein the helical screw is configured to rotate with the at least one wire; and a tubular element threaded over a portion of the length, wherein: the tubular element comprises a tip comprising at least one blade structure; upon rotation of the at least one wire, the helical screw is configured to generate a negative pressure within the tubular element; and the negative pressure within the tubular element is configured to draw material toward the tip to enable cutting of the material against the helical screw and the at least one blade structure.

In some embodiments, a respective blade structure of the at least one blade structure comprises: a rounded external surface; and at least one sharpened interior edge. In some embodiments, the at least one blade structure comprises a plurality of blade structures in a radial arrangement. In some embodiments, the respective rounded external surfaces of the plurality of blade structures define a hemisphere comprising a central void and a plurality of radially spaced voids between respective blade structures. In some embodiments, the tubular element is configured to remain static relative to the rotation of the at least one wire. In some embodiments, the system further comprises a termination cap coupled to the at least one wire at one of the opposed ends. In some embodiments, a connection between the termination cap and the at least one wire comprises at least one polymer material. In some embodiments, a connection between the termination cap and the at least one wire comprises at least one solder material.

In some embodiments, the system further comprises at least one rotation mechanism operatively connected the at least one wire, the at least one mechanism configured to rotate the at least one wire upon activation. In some embodiments, the at least one wire comprises a plurality of wires. In some embodiments, the at least one wire comprises stainless steel. In some embodiments, the at least one wire comprises nitinol. In some embodiments, the at least one wire comprises at least one radiopaque material. In some embodiments, the helical screw comprises stainless steel. In some embodiments, the helical screw comprises tungsten carbide. In some embodiments, the helical screw comprises at least one radiopaque material.

An example kit may comprise one or more atherectomy systems as described herein and shown in the accompanying figures. For example, the kit may include one or more systems as described above. In some embodiments, the kit further includes at least one guidewire, wherein one or more systems of the kit is/are configured to be deployed to a target site via the at least one guidewire. In some embodiments, the kit comprises a first system comprising a respective tubular element of a first bore size; and a second system comprising a respective tubular element of a second bore size, wherein the second bore size exceeds the first bore size.

An example method of use for an atherectomy system (or kit) of the present disclosure may include removing material from a target site within a tubular structure. The example method may include deploying a guidewire to a tubular structure of a subject, the tubular structure comprising a target material; inserting an atherectomy system (such as one or more example systems described above) into the tubular structure via the guidewire; rotating the at least one wire via a rotation mechanism coupled to one of the opposed ends of the at least one wire; and advancing the tip of the helical screw into the target material, wherein: the rotating of the at least one wire causes rotation of the helical screw; the rotation of the helical screw draws the target material to the tip of the tubular element; and the at least one blade structure and the helical screw cut the target material in a region proximate to the tip of the tubular element.

In some embodiments, the cutting of the target material is caused by collective engagement of the target material with the helical screw and the at least one blade structure. In some embodiments, the method further comprises aspirating the target material through the tubular element out of the subject. In some embodiments, the target material comprises at least one clot. In some embodiments, the rotating of the at least one wire is performed at a rate of at least 10,000 revolutions per minute (RPM). In some embodiments, the target material includes plaque, one or more emboli (e.g., thrombus, foreign object, fat globule) and/or the like. In some embodiments, the method further includes retracting the atherectomy system from the tubular structure along the guidewire.

### BRIEF DESCRIPTION OF THE FIGURES

Having thus described the embodiments of the disclosure in general terms, reference now will be made to the accompanying drawings, which are not necessarily drawn to scale, and wherein:

FIG. 1 shows a left perspective view of an example atherectomy system in accordance with some embodiments of the present disclosure;

FIG. 2 shows a right perspective view of an example atherectomy system in accordance with some embodiments of the present disclosure;

FIG. 3 shows a partial perspective view of an example atherectomy system in accordance with some embodiments of the present disclosure;

FIG. 4A shows a front view of an example atherectomy system in accordance with some embodiments of the present disclosure;

FIG. 4B shows a back view of an example atherectomy system in accordance with some embodiments of the present disclosure;

FIG. 5A shows a top view of an example atherectomy system in accordance with some embodiments of the present disclosure;

FIG. 5B shows a bottom view of an example atherectomy system in accordance with some embodiments of the present disclosure;

FIG. 5C shows a left side view of an example atherectomy system in accordance with some embodiments of the present disclosure;

FIG. 5D shows a right side view of an example atherectomy system in accordance with some embodiments of the present disclosure;

FIG. 6 shows a left perspective view of an example tubular element in accordance with some embodiments of the present disclosure;

FIG. 7 shows a right perspective view of an example tubular element in accordance with some embodiments of the present disclosure;

FIG. 8 shows a rear perspective view of an example tubular element in accordance with some embodiments of the present disclosure;

FIG. 9A shows a front view of an example tubular element in accordance with some embodiments of the present disclosure;

FIG. 9B shows a back view of an example tubular element in accordance with some embodiments of the present disclosure;

FIG. 10A shows a left side view of an example tubular element in accordance with some embodiments of the present disclosure;

FIG. 10B shows a right side view of an example tubular element in accordance with some embodiments of the present disclosure;

FIG. 11 shows a perspective view of an example wire structure in accordance with some embodiments of the present disclosure;

FIG. 12 shows a partial perspective view of an example wire structure in accordance with some embodiments of the present disclosure;

FIG. 13 shows a perspective view of an example helical screw in accordance with some embodiments of the present disclosure;

FIG. 14 shows a partial perspective view of an example helical screw in accordance with some embodiments of the present disclosure;

FIG. 15 shows a diagram of an example atherectomy system in accordance with some embodiments of the present disclosure;

FIG. 16 shows a diagram of an example atherectomy system in accordance with some embodiments of the present disclosure;

FIG. 17 shows a diagram of an example atherectomy system in accordance with some embodiments of the present disclosure;

FIG. 18 shows a perspective view of an example atherectomy system in accordance with some embodiments of the present disclosure;

FIG. 19 shows a partial perspective view of an example atherectomy system in accordance with some embodiments of the present disclosure; and

FIG. 20 illustrates a flowchart of an example atherectomy process in accordance with some embodiments of the present disclosure.

### DETAILED DESCRIPTION

Some embodiments of the present invention will now be described more fully hereinafter with reference to the accompanying drawings, in which some, but not all, embodiments of the invention are shown. Like reference numerals refer to like elements throughout. Indeed, various embodiments of the invention may be embodied in many different forms and should not be construed as limited to the embodiments set forth herein. Rather, these embodiments are provided so that this disclosure will satisfy applicable legal requirements.

As used herein, the term “or” is used in both the alternative and conjunctive sense, unless otherwise indicated. The term “along,” and similarly utilized terms, means near or on, but not necessarily requiring directly on an edge or other referenced location. The terms “approximately,” “generally,” and “substantially” refer to within manufacturing and/or engineering design tolerances for the corresponding materials and/or elements unless otherwise indicated. Thus, use of any such aforementioned terms, or similarly interchangeable terms, should not be taken to limit the spirit and scope of embodiments of the present invention.

As used herein, reference is made to a system for performing an atherectomy procedure, which may include engaging and removing material from a tubular structure. The present disclosure, however, contemplates that the systems and methods of the present disclosure may be equally applicable to other applications in which reduced diameter cutting systems are advantageous. For example, the atherectomy system may be used in other material extraction procedures, such as embolectomy, intestinal obstruction removal, and/or the like.

As used herein, the terms “atherectomy system” and “auger wire” may be used interchangeably to a system configured to disrupt and remove material from a tubular structure.

### Overview

In general, various embodiments of the present disclosure provide improved designs for atherectomy systems. For example, the disclosure provides various embodiments for an atherectomy system that may be deployed via catheter to a tubular structure and rotated to cut and remove target material, such as plaque. The various atherectomy systems described herein and shown in the figures may demonstrate reduced spatial profiles as compared to conventional systems for removing material from a tubular structure. In doing so, the present atherectomy system may overcome challenges associated with removing material from small-diameter blood vessels. It will be understood and appreciated that such context is provided by way of example and uses of the atherectomy system in additional contexts, such as with other medical procedures, are contemplated and within the scope of the invention.

As described above, existing atherectomy systems face challenges in effectively and safely removing target material from small diameter tubular structures, such as narrow arteries and veins. For example, the miniaturization of laser- and/or sonic-based atherectomy systems may be infeasible. As another example, the use of front- and side-bladed directional atherectomy systems in small diameter blood vessels may introduce an increased risk of cutting tissue. In still another example, minimization of burr- or drill-based rotational atherectomy systems may be impractical due to constraints in motor dimension, steering complexity, and/or the like.

To solve these issues and others, example implementations of embodiments of the present application may provide an atherectomy system comprising a torqueable wire with a helically wrapped wire used to transmit rotational movement at high speeds (e.g., equal to or greater than 10,000 RPM). In various embodiments, when rotated, the helically wrapped wire (“helical screw”) may transport target material from a tubular structure into the tip of a tubular element. The wire-based means for detaching and transporting target material may support navigability of the atherectomy system in small diameter tubular structures.

One or more blade structures at the tip of the tubular element may engage with the helical screw to cut the target material in a scissor-like action as the helical screw pulls the target material against the blade structure. The blade structure may include a sharpened interior edge and rounded and/or blunted external edges and surfaces. The internalization of sharp edges may reduce a likelihood of damaging tissue. For example, in contrast to existing approaches that cut or drill target material externally within the tubular structure, the present atherectomy system may cut target material internally such that sharp edges and surfaces are not exposed to the tubular structure. The rounded external surfaces and blunted external edges of the tubular element may reduce a likelihood of piercing or tearing tissue as the atherectomy system is advanced through a tubular structure.

In this manner, the atherectomy system described hereafter improves safety and feasibility of removing target material from a small diameter blood vessel or other tubular structure. The internalization of cutting edges within the system may reduce a likelihood of damaging tissues. Further, the helical screw and wire-based means for engaging and transporting target material improve steerability of the atherectomy system through tortuous pathways. In doing so, the atherectomy system may achieve adequate debulking of target materials from small diameter environments while providing measures for mitigating risk of complications.

### Example Atherectomy System

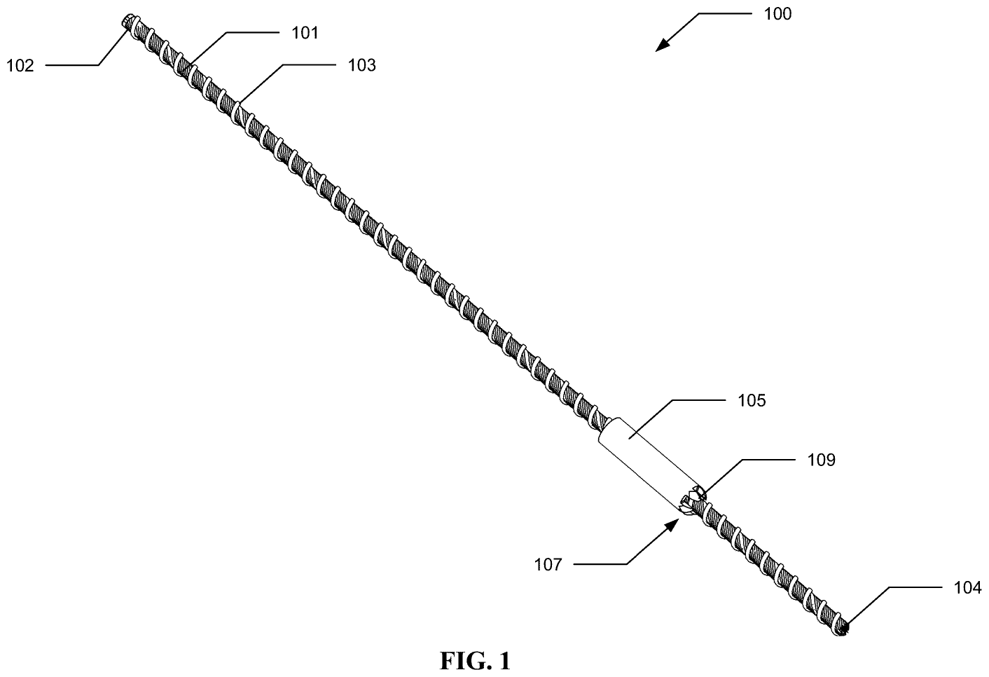

With reference to FIG. 1, shown is a left perspective view of an example atherectomy system 100. In some embodiments, the atherectomy system 100 includes one or more wires 101, a helical screw 103 attached along a length of the wire 101, and a tubular element 105 through which the wire 101 and helical screw 103 may be inserted. In various embodiments, the one or more wires 101 and helical screw 103 are configured to rotate together in response to a torque applied to the wire 101. In some embodiments, the tubular element 105 is configured to remain static relative to rotation of the helical screw 103 and one or more wires 101.

In some embodiments, the wire 101 includes opposed ends 102, 104 that define a length of the wire 101. In some embodiments, the wire 101 is attachable to a rotation mechanism that torques (e.g., rotates) the wire 101 at a rotation speed of 10,000 revolutions per minute (RPM) or greater. For example, a chuck mechanism may secure to and connect the wire 101 to a rotation mechanism such that the wire 101 may be torqued via activation of the rotation mechanism. In some embodiments, the atherectomy system 100 includes a plurality of wires 101. For example, the atherectomy system 100 may include a plurality of wires 101 that may be torqued as a singular assemblage. In some embodiments, the plurality of wires 101 are attached to one another via soldering, laser welding, adhesion, and/or the like such that the wires 101 may be rotated together. For example, a plurality of wires 101 may be attached to one another such that the wires 101 may be collectively torqued. In some embodiments, respective wires 101 are bonded to one another along a subset of their total lengths. For example, a plurality of wires 101 may be welded, adhered, or otherwise bonded to one another at one or more segments, and a remaining subset of the respective wires may be unbonded.

In some embodiments, the wire 101 includes stainless steel, nitinol, and/or the like. In some embodiments, the wire 101 includes one or more radiopaque materials. For example, the wire 101 may include one or more radiopaque markers, such as barium sulfate markers or other radiopaque contrast media. In some embodiments, the wire 101 includes a circular cross-section. Alternatively, in some embodiments, the wire 101 includes an elliptical cross-section. In some embodiments, a plurality of interconnected wires 101 demonstrate varying cross-sections. For example, a plurality of wires 101 may include a first subset of wires having circulate cross-sections and a second subset of wires having elliptical cross-sections.

In some embodiments, the helical screw 103 includes a wire comprising a helical shape. In some embodiments, the helical screw 103 includes an elliptical cross-section. Alternatively, in some embodiments, the helical screw 103 includes a circular cross-section. In various embodiments, the helical screw 103 is coupled to the one or more wires 101 along the length of the one or more wires 101 between the opposed ends 102, 104. For example, the helical screw 103 may be connected to the wire 101 via soldering, laser welding, adhesives, and/or the like. In some embodiments, the helical screw 103 is configured to rotate with the one or more wires 101. For example, the helical screw 103 may be rotated via torque applied to an end 102 of the one or more wires 101. In some embodiments, upon rotation of the one or more wires 101, the helical screw 103 is configured to generate a negative pressure within the tubular element 105, which may draw material toward and into the tubular element 105. As further described herein, the helical screw 103 and one or more blade structures 109 of the tubular element 105 may cut the material. For example, the helical screw 103 and a blade structure 109 may cut material via a collective engagement between the helical screw 103 and a sharpened interior edge of the blade structure in a scissor-like manner. In some embodiments, the helical screw 103 includes stainless steel, tungsten carbide, and/or the like. In some embodiments the helical screw 103 includes one or more radiopaque materials. For example, the helical screw 103 may include one or more markers including barium sulfate or other radiopaque contrast media. In some embodiments, the helical screw 103 is integrally formed with one or more wires 101.

In some embodiments, the tubular element 105 includes a cylindrical shape. In some embodiments, an end of the cylindrical shape includes a hemisphere (e.g., half-sphere) shape embodying a tip 107 of the tubular element 105. The tubular element 105 may include a central void through which the helical screw 103 and one or more wires 101 may be inserted. In some embodiments, the tip 107 includes one or more blade structures 109. In some embodiments, a blade structure 109 is configured to cut material proximate to the tip 107 via engagement between the blade structure 109 and the helical screw 103. In some embodiments, the tip 107 includes a plurality of blade structures 109 in a radial arrangement. The plurality of blade structures 109 may be spaced apart from one another in the radial arrangement such that gaps are present between adjacent blade structures. In some embodiments, the tubular element 105 includes titanium, one or more titanium-comprising alloys, tungsten carbide, and/or the like. In some embodiments, the tubular element 105 includes one or more radiopaque materials.

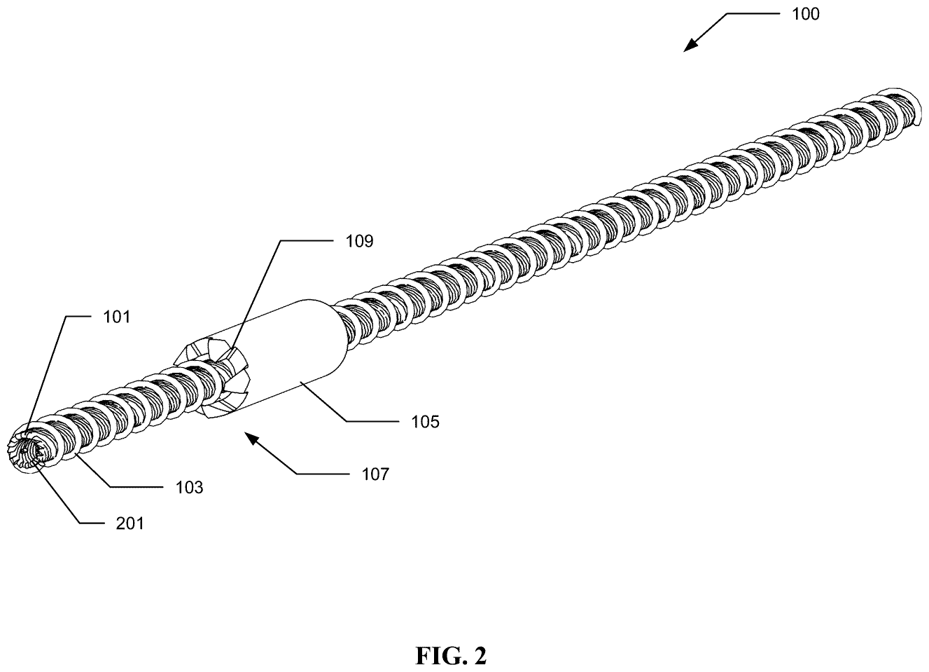

FIG. 2 shows a right perspective view of an example atherectomy system 100. As shown, the atherectomy system 100 may include a plurality of wires 101 arranged into a spiral structure. The helical screw 103 may be coupled to the spiral structure. For example, the helical screw 103 may embody one or more wires attached around the spiral structure.

In some embodiments, the plurality of wires 101 include a central void 201. In some embodiments, the central void 201 is configured to receive a guidewire and/or the like for navigating the atherectomy system 100 to a target site of a tubular structure. In some embodiments, the one or more wires 101 and helical screw 103 are flexible such that the atherectomy system 100 may be navigated through a tortuous environment. For example, the helical screw 103 and a plurality of wires 101 may be configured to bend or contort during navigation of the atherectomy system 100 through one or more blood vessels.

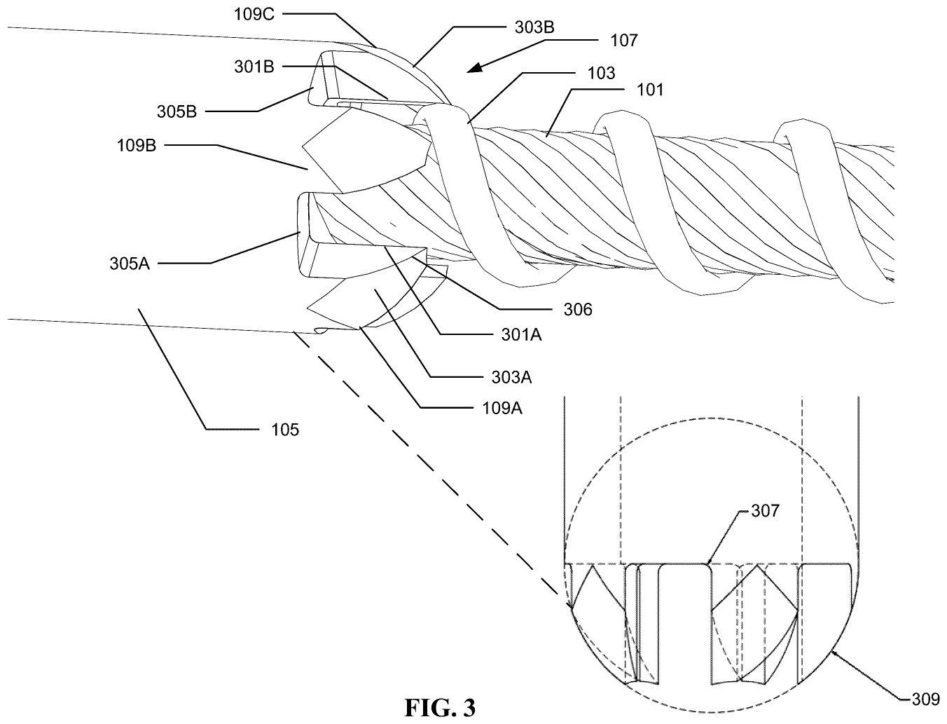

FIG. 3 shows a partial perspective view of an example atherectomy system 100. In various embodiments, the tip 107 includes a plurality of blade structures. For example, the tip 107 may include a plurality of blade structures 109A, 109B, 109C in a radial arrangement. In some embodiments, a respective blade structure includes an interior edge that is sharpened such that the interior edge may engage with the rotating helical screw 103 to cut material proximate to the tip 107. For example, the blade structures 109A, 109B, 109C may include interior edges 301A, 301B, 301C, respectively, where each interior edge is configured to engage with the helical screw 103 to cut material drawn toward the tip 107 of the tubular element 105. In some embodiments, a respected blade structure includes a rounded external surface including blunted outer edges. For example, the blade structures 109A, 109B, 109C may include external surfaces 303A, 303B, 303C that are rounded and comprise blunted outer edges 306. In various embodiments, the rounded surfaces define a hemisphere shape of the tip 107. In some embodiments, the hemisphere includes a central void, a plurality of blade structures in radial arrangement and a plurality of radially spaced voids between adjacent blade structures. For example, the blade structure 109A and blade structure 109B may be spaced apart in the radial arrangement such that a void 305A is present between the adjacent blade structures. Further, the blade structure 109B and blade structure 109C may be spaced apart in the radial arrangement such that a void 305B is presented between the blade structures.

In some embodiments, the blunted outer edges of the rounded external surfaces include a radius 307 that transitions the outer edge to a surfacing defining the gap between adjacent blade structures. In some embodiments, the radius 307 is 0.0254 mm. In some embodiments, the blunted outer edges of the rounded external surfaces include a radius 309. In some embodiments, the radius 309 ranges between 0.005 millimeters (mm) and 0.7 mm. For example, the radius 309 may be 0.406 mm.

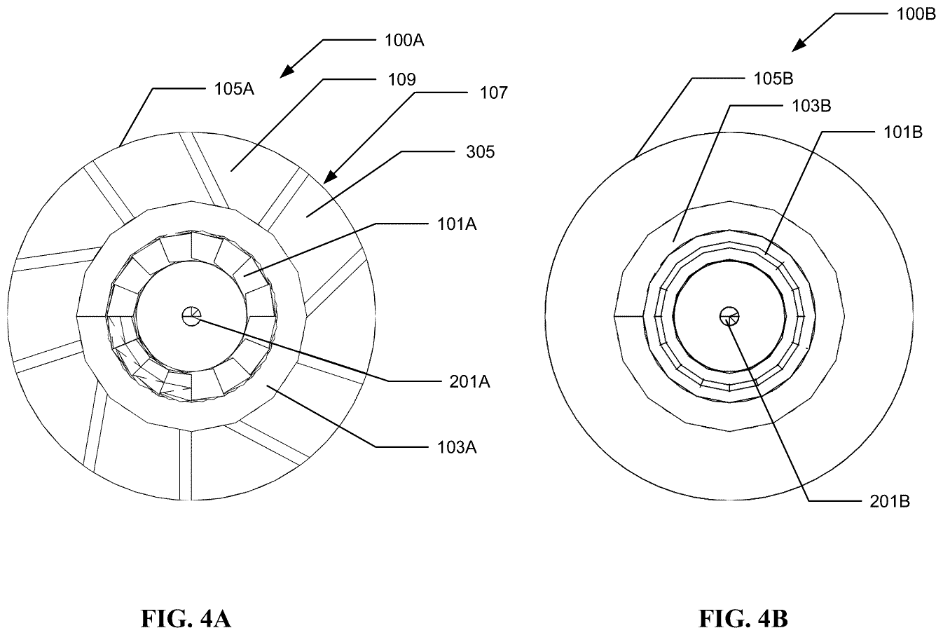

FIG. 4A shows a front view of an example atherectomy system 100A. The tip 107 of the tubular element 105A may include any suitable number of blade structures (e.g., 1, 3, 5, 6, or any other suitable number). For example, as shown in FIG. 4A, the tubular element 105A may include five blade structures 109 radially arranged and spaced apart from one another by five voids 305. In some embodiments, the voids 305 enable entry of material from the sides of the tubular element 105 into the tip 107 (e.g., within which the blade structures, together with the helical screw 103, may cut the material). FIG. 4B shows a back view of an example atherectomy system 100B. As shown in FIGS. 4A and 4B, the atherectomy system 100A, 100B may include a central void 201A, 201B that extends longitudinally through the plurality of wires 101, helical screw 103, and tubular element 105.

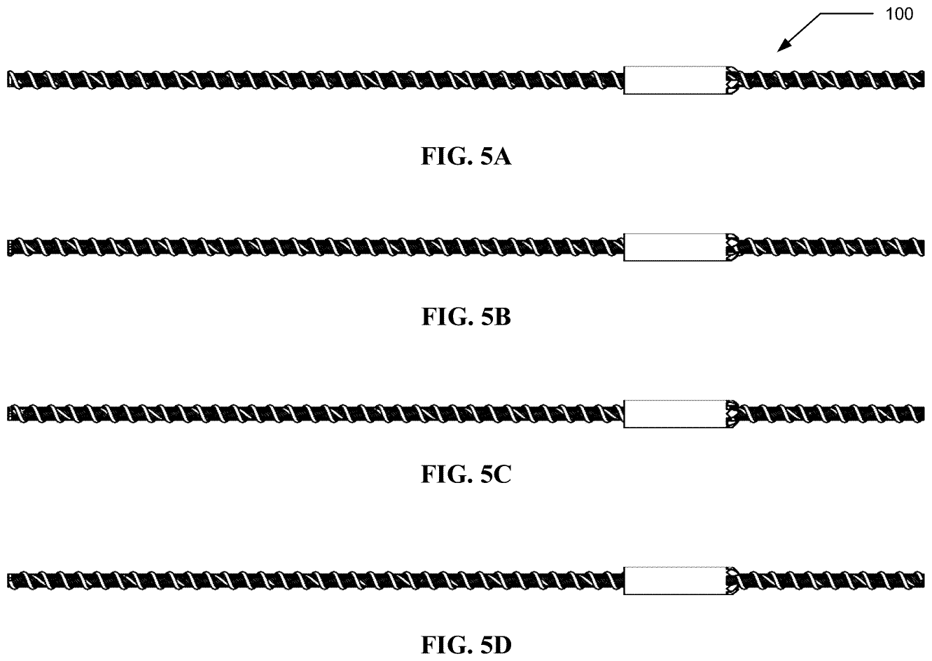

FIG. 5A shows a top view of an example atherectomy system 100.

FIG. 5B shows a bottom view of the example atherectomy system 100.

FIG. 5C shows a left side view of the example atherectomy system 100.

FIG. 5D shows a right side view of the example atherectomy system 100.

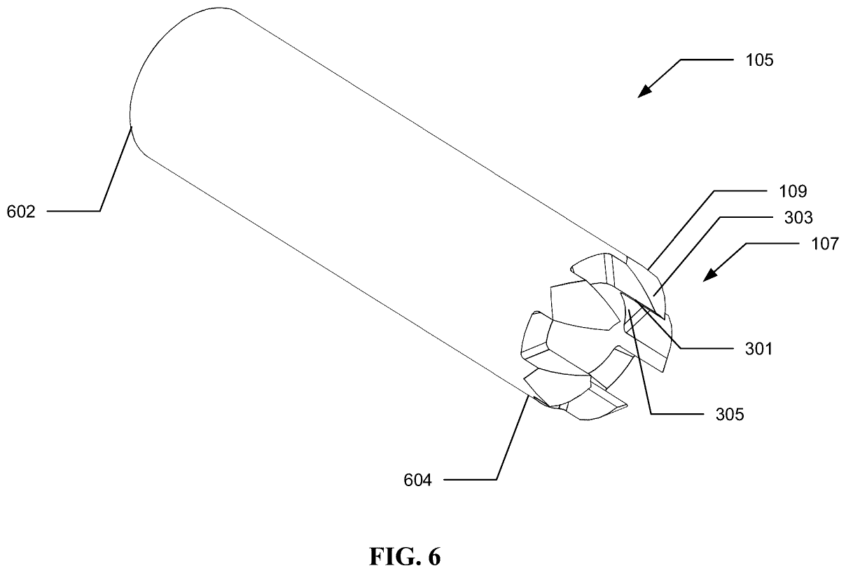

FIG. 6 shows a left perspective view of an example tubular element 105. As shown, the tubular element 105 comprises a generally cylindrical shape between a first end 602 and a second end 604. In various embodiments, the tip 107 of the tubular element 105 extends from the second end 604 and comprises a plurality of blade structures 109 that define a hemisphere shape.

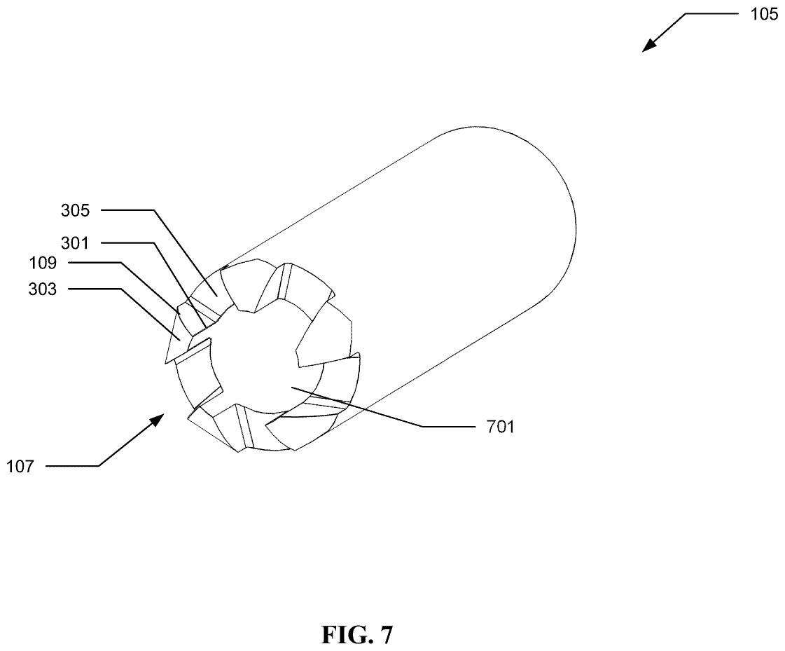

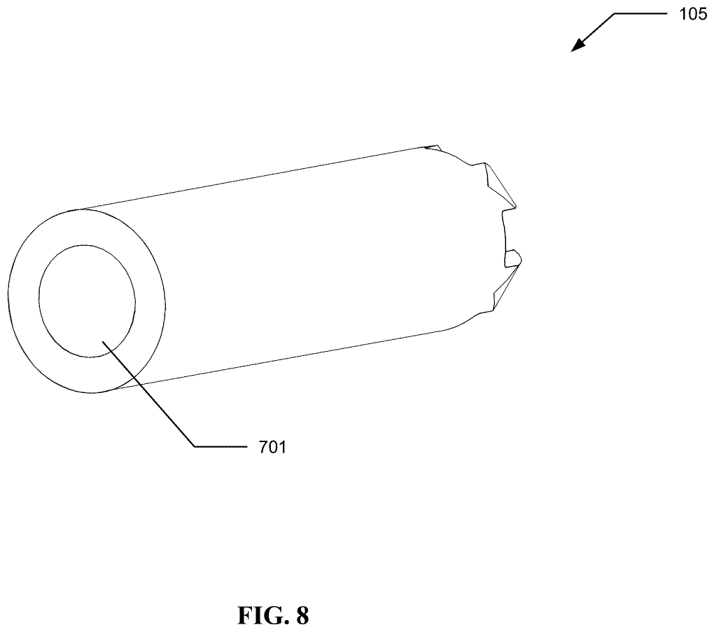

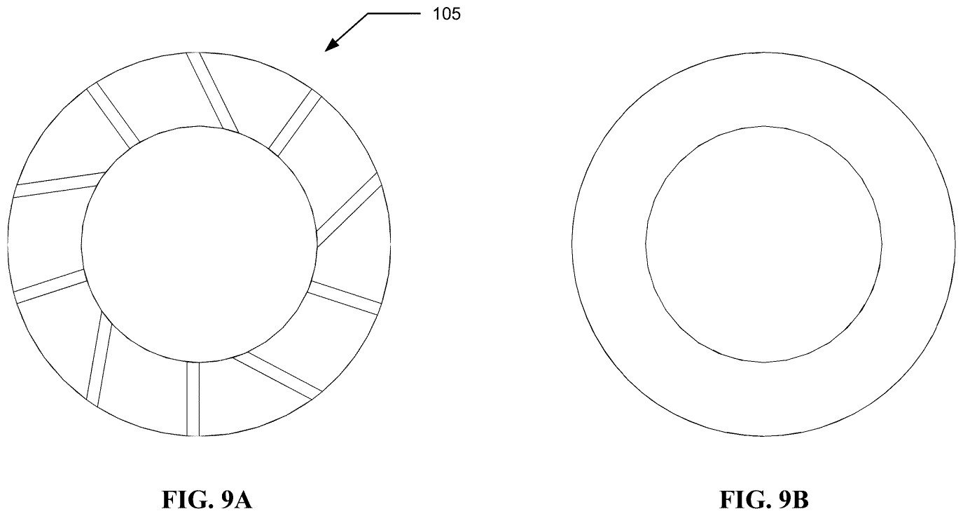

FIG. 7 shows a right perspective view of an example tubular element 105. In various embodiments, a material that enters the tip 107 via the voids 305 (or open tip front) may be cut via engagement of the interior edge 301 with a helical screw (not shown). In some embodiments, the cut material may be further drawn (under negative pressure, screw movement, and/or the like) into a central void 701 of the tubular element 105. As further shown in the rear perspective view of FIG. 8, the central void 701 may extend longitudinally through the tubular element 105. FIG. 9A shows a front view of an example tubular element 105.

FIG. 9B shows a back view of the example tubular element 105.

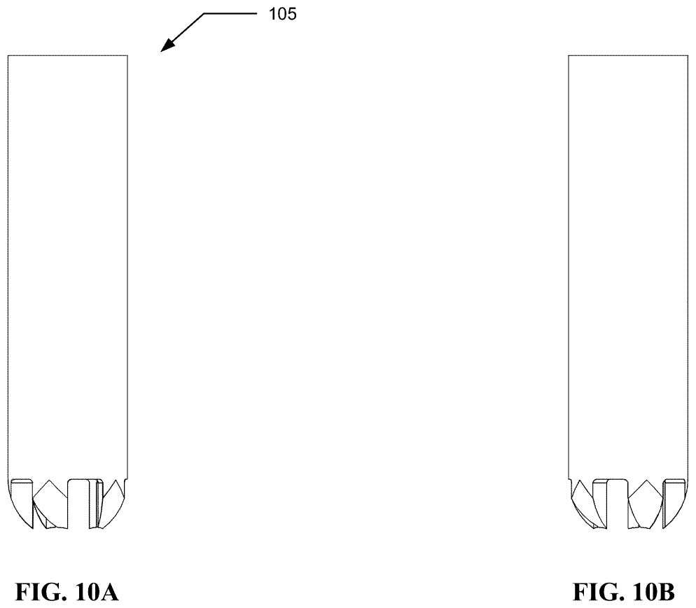

FIG. 10A shows a left side view of the example tubular element 105.

FIG. 10B shows a right side view of the example tubular element 105.

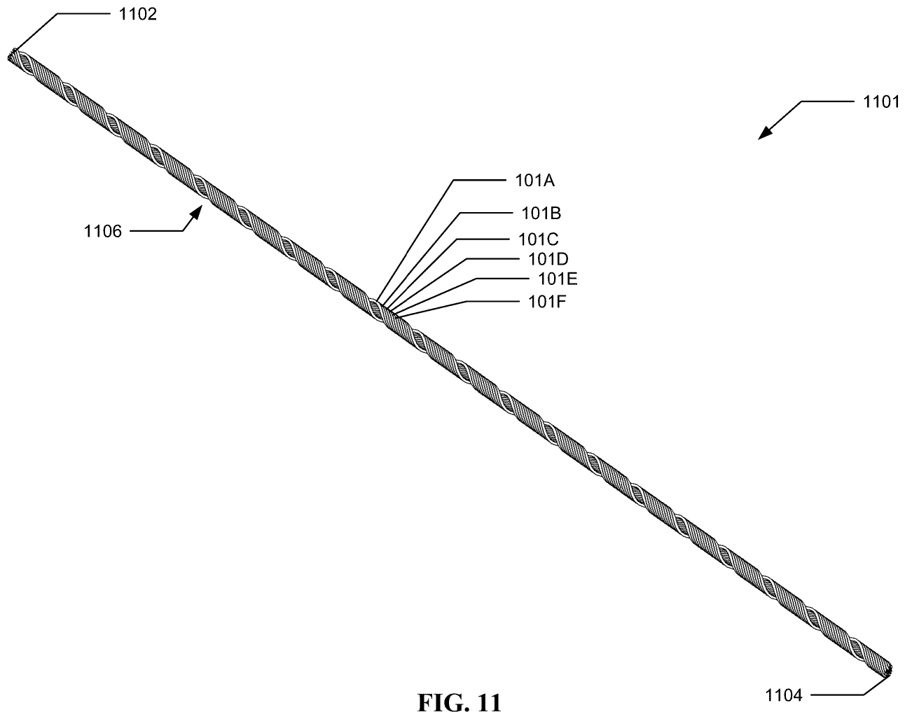

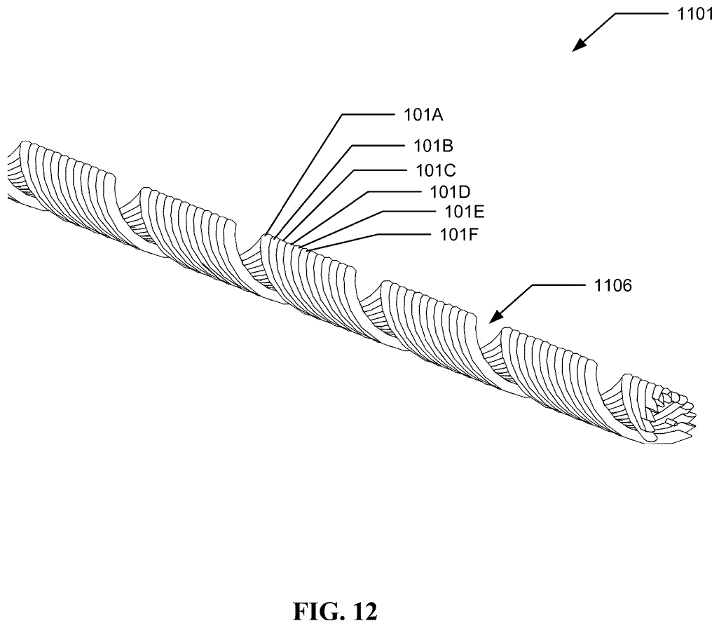

FIG. 11 shows a perspective view of an example wire structure 1101. In some embodiments, the atherectomy system of the present disclosure includes a wire structure 1101 comprising a plurality of wires arranged in a spiral shape. For example, the wire structure 1101 may include a plurality of wires 101A, 101B, 101C, 101D, 101E, 101F (or greater number) that are at least partially bonded to one another and wound into a spiral shape. In some embodiments, the wire structure 1101 includes a central void (see, for example, central void 201 shown in FIG. 2 and described herein). Additionally, in some embodiments, the wire structure 1101 includes a helical channel 1106 configured to receive a portion of a helical screw (not shown). For example, a helical screw may be arranged within the helical channel 1106 and coupled to one or more wires embodying the wire structure 1101 (e.g., via soldering, laser welding, adhesives, and/or the like). The partial perspective view of FIG. 12 further illustrates the wire structure 1101 and helical channel 1106 formed along the length of the wire structure 1101.

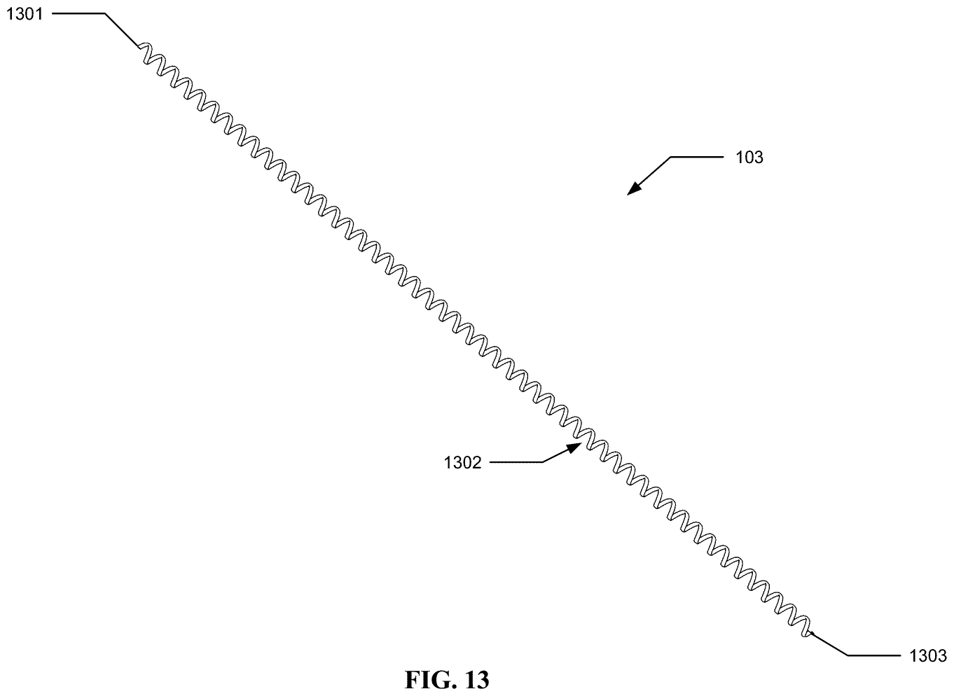

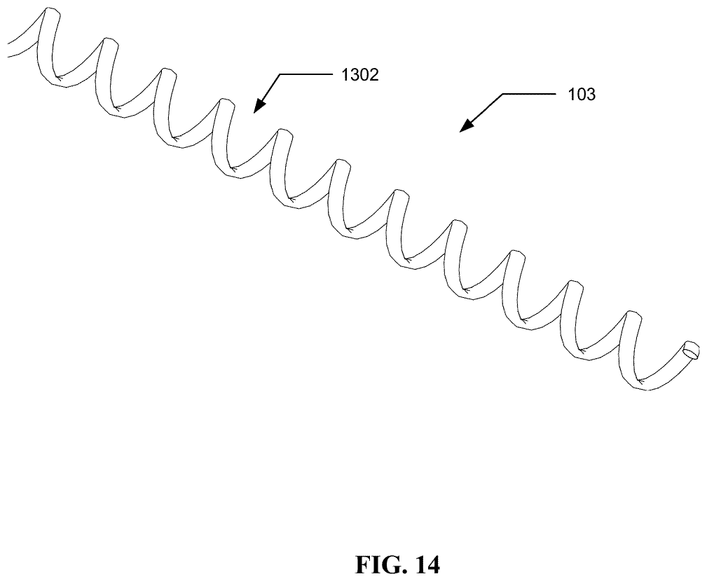

FIG. 13 shows a perspective view of an example helical screw 103. In some embodiments, the helical screw 103 embodies a spiral-shaped wire extending between opposed ends 1301, 1303. The helical screw 103 may embody a right-handed or left-handed spiral shape. In various embodiments, the helical screw 103 includes a central void 1302 through which a guidewire and/or the like may be inserted. The partial perspective view of FIG. 14 further illustrates the central void 1302 of the helical screw 103.

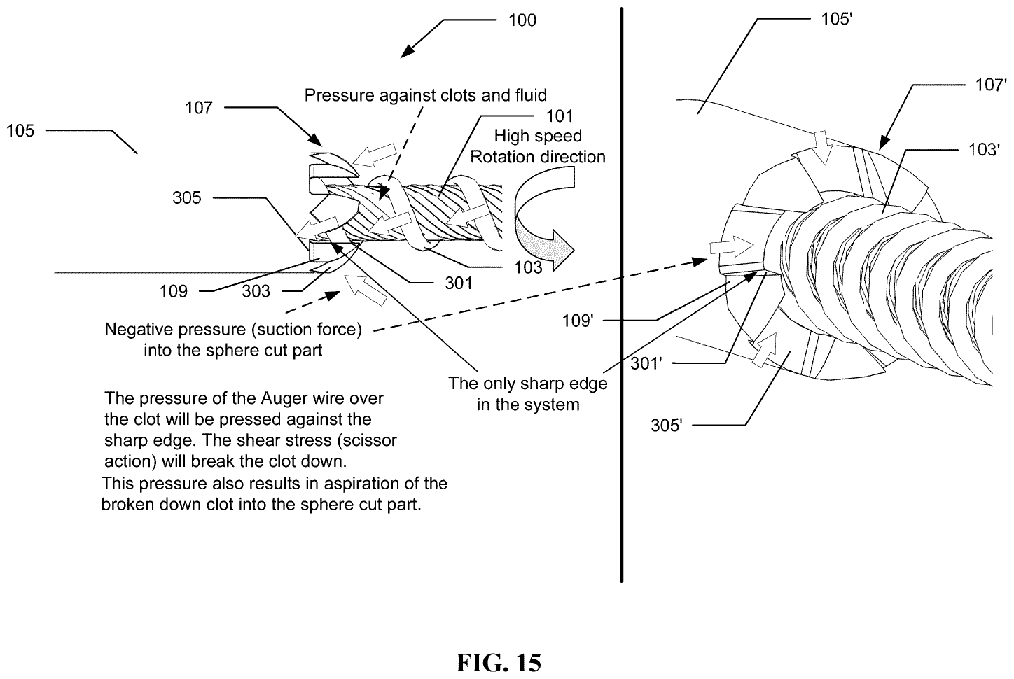

FIG. 15 shows a diagram of an example atherectomy system 100. In some embodiments, the helical screw 103, 103′ and one or more wires 101 may be rotated at high speed to generate a pressure gradient in a direction toward the tip 107, 107′ of the tubular element 105, 105′. The tubular element 105, 105′ may remain static relative to the rotation of the helical screw 103 and wire 101. The rotation of the helical screw 103, 103′ and wire 101 may further generate a negative pressurization within the tubular element 105, 105′. In various embodiments, the negative pressure suctions material toward and into the tip 107, 107′. In some embodiments, the respective interior edge 301, 301′ of the plurality of blade structures 109, 109′ engage with the rotating helical screw 103, 103′ and, thereby, apply a shear stress (e.g., scissor action) that cuts the material into smaller portions. In some embodiments, the negative pressure causes further aspiration of the cut material into the tubular element 105, 105′ and, potentially, into a collection catheter and/or the like for extracting the material out of a subject.

In various embodiments, by providing sharp edges only on the interior portions of the blade structures 109, the atherectomy system 100 may reduce a likelihood of damaging tissue or other structures of a subject. Further, the rounded external surfaces 303 of the blade structures 109 may reduce a likelihood of piercing tissue or other structures of a subject that may come into contact with the tip 107. In doing so, the atherectomy system 100 may overcome challenges associated with safely disrupting and removing material from small diameter, tortuous tubular structures, such as blood vessels.

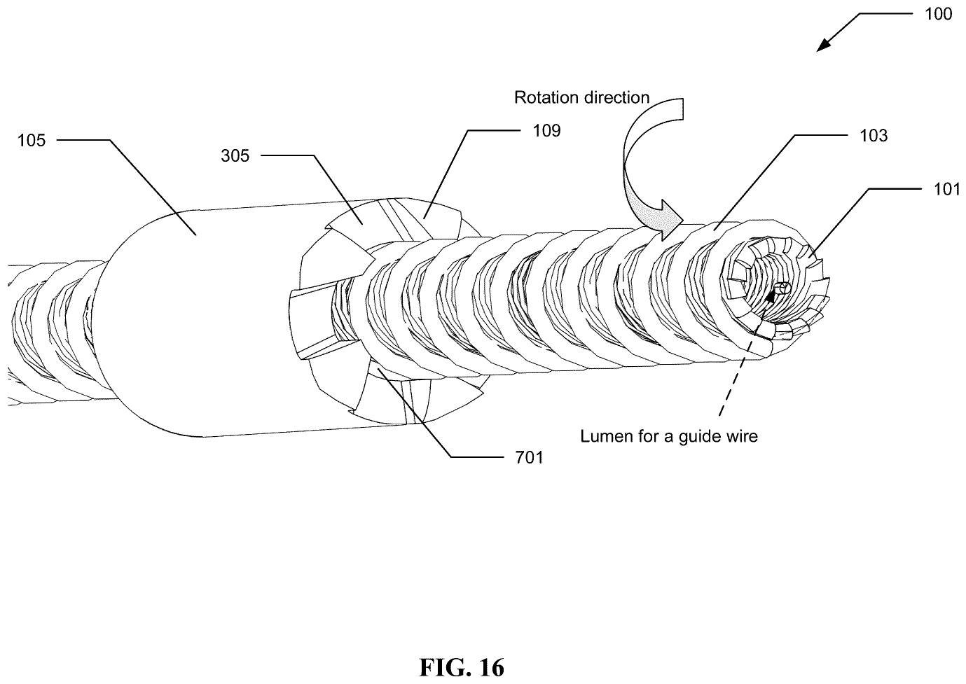

FIG. 16 shows a diagram of an example atherectomy system 100. The one or more wires 101 may be rotated in a first direction to cause rotation of the attached helical screw 103 in the first direction. The rotation of the wire 101 and helical screw 103 may generate a pressure gradient (e.g., negative pressure) into toward the tip 107 and central void 701 of the tubular element 105. In some embodiments, material is drawn into the tip 107 through the exposed front. Additionally, or alternatively, in some embodiments, material is drawn into the tip 107 through the voids 305 between the radially arranged blade structures 109.

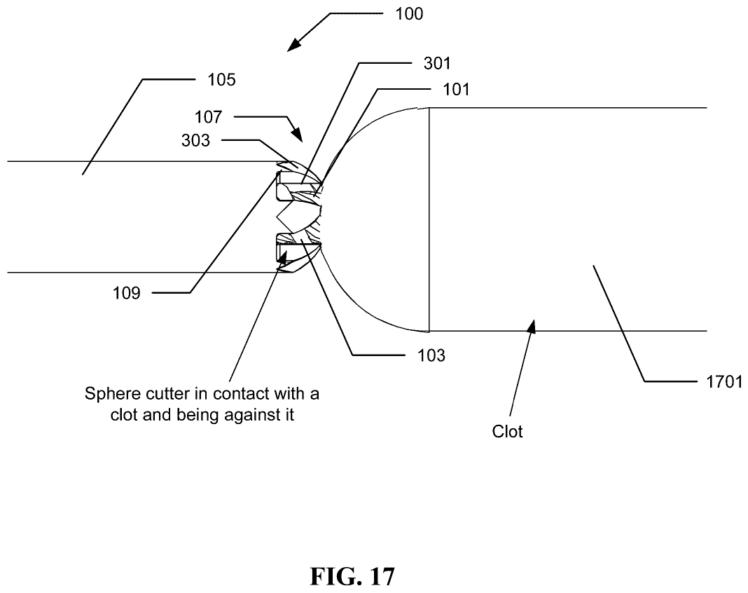

FIG. 17 shows a diagram of an example atherectomy system 100. In some embodiments, a portion of the one or more wires 101 and helical screw 103 are inserted through a target material 1701. The target material 1701 may include plaque, a thrombus, foreign debris, and/or the like. In various embodiments, the wire 101 and helical screw 103 are advanced into the target material 1701 to bring the tip 107 of the tubular element 105 into contact with the target material 1701. In some embodiments, the tip 107 is biased (e.g., pushed) against the target material 1701 such that the one or more blade structures 109 maintain contact with the target material 1701 throughout cutting. In some embodiments, the wire 101 and helical screw 103 are rotated at a high rate of speed (e.g., 10,000 RPM, 20,000 RPM, or another suitable value) to suction the target material 1701 into the tip 107 at which the helical screw 103 and interior edge 301 of the one or more blade structures 109 collectively engage to cut the target material 1701.

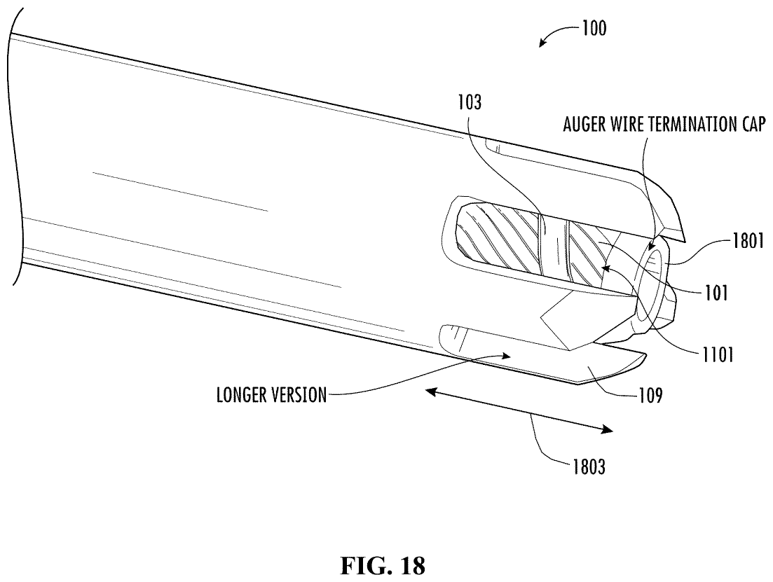

FIG. 18 shows a perspective view of an example atherectomy system 100. In some embodiments, a wire structure 1101 comprised of a plurality of wires 101 includes a termination cap 1801. In some embodiments, the termination cap 1801 is configured to reduce a likelihood of one or more wires 101 piercing or otherwise damaging tissue or other structures of a subject. For example, the termination cap 1801 may provide a barrier between wire ends and the subject. In some embodiments, the termination cap 1801 includes one or more polymers, metal materials, and/or the like. For example, the termination cap 1801 may include solder material comprised of one or more metals.

In some embodiments, a depth 1803 of the blade structure 109 is increased to expose a longer portion of the helical screw 103 to material within a tubular structure. By exposing a longer portion of the helical screw to fluid, plaque, emboli, and/or the like within the tubular structure, the increased depth 1803 may compensate for reduced suctioning pressure in instances of reduced rotation speed. In some embodiments, the depth 1803 of the blade structure 109 may be configured to accommodate various types, dimensions, and/or volumes of target material. For example, in a context of removing low-density, non-calcified plaques, a blade structure 109 having a greater depth 1803 may be utilized as compared to a blade structure of lower depth, which may be utilized in a context of removing calcified plaques. As another example, a blade structure 109 of greater depth 1803 may be utilized in instances of removing plaque of larger cross-sectional area.

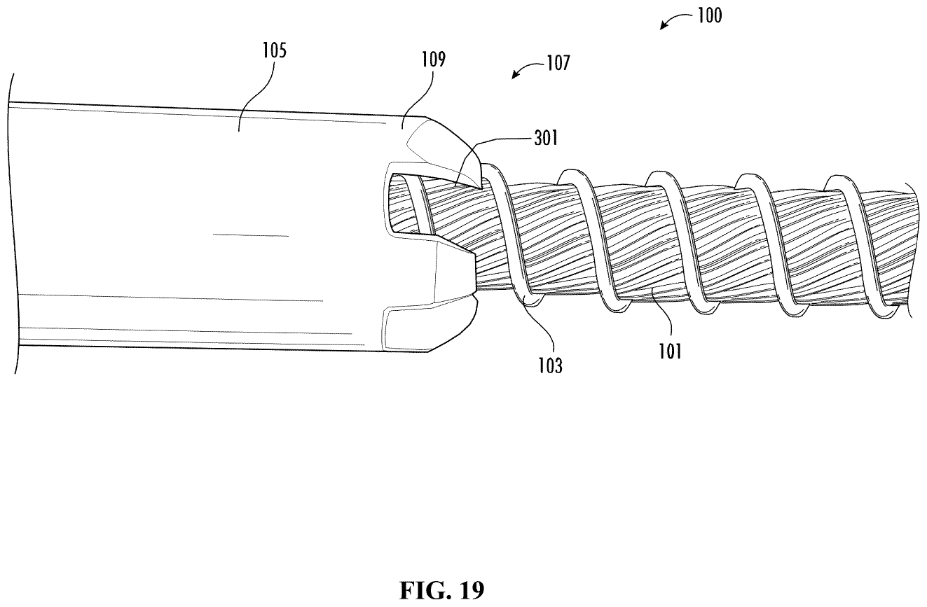

FIG. 19 shows a partial perspective view of an example atherectomy system 100. In some embodiments, the helical screw 103 embodies one or more wires coupled around a plurality of wires 101. The helical screw 103 and interior edges 301 of the respective blade structure 109 may collectively engage to cut target material in a region proximate to the tip 107. In various embodiments, the plurality of wires 101 are operatively connected to a rotation mechanism via one or more clamps. For example, a clamp having radial symmetry (e.g., a chuck, and/or the like) may be secured over a respective end of the plurality of wires 101. A motor may rotate the clamp to apply torque to the plurality of wires 101.

### Example Method of Use

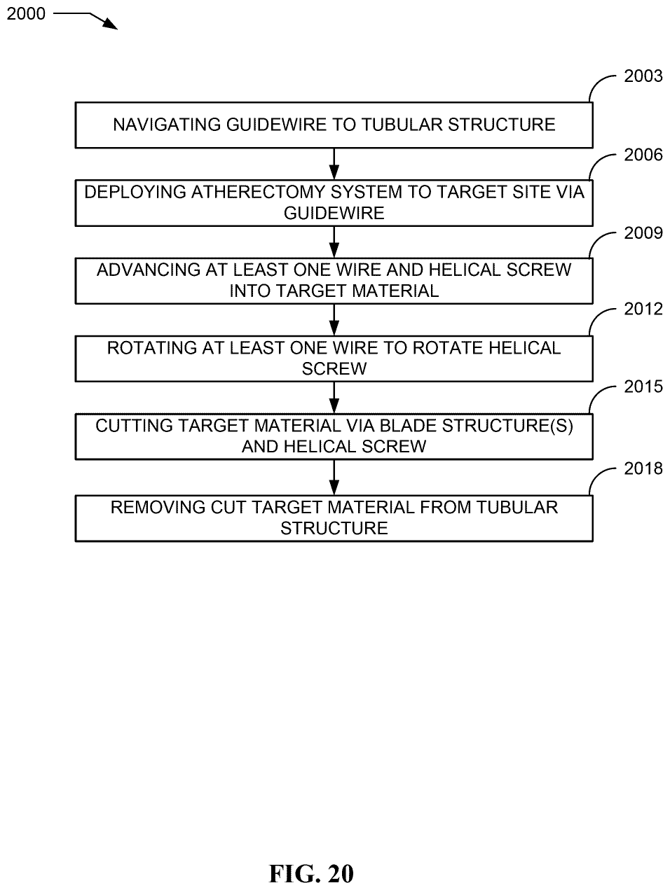

Having described example atherectomy systems in accordance with the disclosure, example processes of the disclosure will now be discussed. It will be appreciated that the flowchart depicts an example process that is performable using one or more of the atherectomy systems described herein. For example, an atherectomy process 2000 depicted in the flowchart of FIG. 20 and described herein may be performed using one or more atherectomy systems 100 as shown in FIGS. 1-5 and 15-19 and described herein. In some embodiments, one or more processes are performed using a kit comprising one or more atherectomy systems. For example, the atherectomy process 2000 may be performed using a kit comprising a first atherectomy system 100 and a second atherectomy system 100. The first atherectomy system 100 may include a tubular element 105 comprising a first bore size, and the second atherectomy system 100 may include a tubular element 105 comprising a second bore size. The second bore size may be less than, greater than, or equal to the first bore size. In some embodiments, one of a plurality of atherectomy systems from a kit may be utilized based at least in part on a diameter of a target tubular structure, target material, and/or the like. In some embodiments, the kit further includes one or more guidewires configured to be inserted into a subject and navigated to a target site to enable deployment of an atherectomy system to the target site via the guidewire.

In some embodiments, the kit comprises one or more connection mechanisms configured to connect an atherectomy system to a rotation mechanism. In some embodiments, the kit further comprises a rotation mechanism. For example, a kit may include a motorized drill and a clamp. The clamp may be secured over a plurality of wires 101 of an atherectomy system 100 to enable rotation of the plurality of wires 101 and helical screw 103 via the motorized drill.

The depicted blocks indicate operations of each process. Such operations may be performed in any of a number of ways, including, without limitation, in the order and manner as depicted and described herein. In some embodiments, one or more blocks of any of the processes described herein occur in-between one or more blocks of another process, before one or more blocks of another process, in parallel with one or more blocks of another process, and/or as a sub-process of a second process. Additionally, or alternatively, any of the processes in various embodiments include some or all operational steps described and/or depicted, including one or more optional blocks in some embodiments. With regard to the flowcharts illustrated herein, one or more of the depicted block(s) in some embodiments is/are optional in some, or all, embodiments of the disclosure. It should be appreciated that one or more of the operations of each flowchart may be combinable, replaceable, and/or otherwise altered as described herein.

FIG. 20 illustrates a flowchart depicting operations of an example atherectomy process 2000 for disrupting and removing a target material from a tubular structure. For example, the atherectomy process 2000 may be performed to degrade and remove plaque from a blood vessel.

In some embodiments, at block 2003, the atherectomy process 2000 includes navigating a guidewire to a tubular structure of a subject. For example, a guidewire may be inserted into an artery of a subject via an incision and navigated through the artery to a particular section thereof or to a secondary blood vessel (e.g., other artery, vein, and/or the like). The tubular structure may include one or more target materials, such as plaque, emboli, and/or the like.

In some embodiments, at block 2006, the atherectomy process 2000 includes deploying an atherectomy system to the tubular structure via the guidewire. For example, an atherectomy system 100 may be deployed to a target tubular structure over an inserted guidewire. Alternatively, in some embodiments, the atherectomy system 100 is navigated through the subject to the target tubular structure without use of a guidewire. In some embodiments, the position of the guidewire, atherectomy system, and/or the like may be determined via one or more medical imaging techniques. For example, the position of the atherectomy system may be obtained via fluoroscopy, x-ray, computed tomography (CT), sonography, positron emission tomography (PET), and/or the like.

In some embodiments, at block 2009, the atherectomy process 2000 includes advancing at least one wire and a helical screw (e.g., coupled around the at least one wire) into target material at the tubular structure. For example, a plurality of wires 101 secured to one another and a helical screw 103 coupled around the plurality of wires 101 may be inserted into plaque, an embolus, and/or the like. In some embodiments, insertion of the helical screw and one or more wires is verified using one or more medical imaging techniques. For example, radiopaque markers on the helical screw, wire, and/or the like may be imaged via fluoroscopy or x-ray to verify the position of the atherectomy system relative to the subject and target material. In some embodiments, the helical screw and one or more wires are inserted into the target material such that the tip of the tubular element of the atherectomy system is pressed against the target material. For example, as shown in FIG. 17, the helical screw 103 and one or more wires 101 may be inserted into the clot such that the tip 107 of the tubular element 105 is in contact with the clot.

In some embodiments, at block 2012, the atherectomy process 2000 includes rotating the at least one wire to rotate the helical screw. For example, a plurality of wires 101 may be rotated to cause rotation of a helical screw 103 coupled to the plurality of wires 101. In some embodiments, the atherectomy process 2000 includes connecting a rotation mechanism to an end of the one or more wires (e.g., a second end of the wires that is opposite a first end, which is inserted into the target material.) The respective ends of the one or more wires may be external to the subject. In some embodiments, the wire and helical screw are rotated at a rate of at least 10,000 RPM. Additionally, or alternatively, in some embodiments, the wire and helical screw are rotated at a rate of at least 20,000 RPM. In some embodiments, the position of the wire and helical screw relative to the target material is adjusted throughout performance of blocks 2012-2018. For example, the wire and helical screw may be further advanced into the target material during performance of block 2015. In various embodiments, the rotation of the helical screw generates a pressure gradient into the tip of the tubular element of the atherectomy system. For example, the rotation of the helical screw 103 may generate a negative pressure that suctions target material into the tip 107 of the tubular element 105.

In some embodiments, at block 2015, the atherectomy process 2000 includes cutting the target material via collective engagement of the helical screw and one or more blade structures of the tubular element of the atherectomy system. For example, the helical screw 103 and interior edge 301 of the blade structure 109 may collectively engage to cut the target material as the target material passes through the tip 107 of the tubular element 105. The helical screw 103 may pull the target material against the interior edge of the blade structure, which may result the target being cut in a scissor-like manner. In some embodiments, the external edges and surfaces of the blade structures are rounded and/or blunted to reduce a likelihood of cutting or piercing the tubular structure or other tissues of the subject. In various embodiments, an end of the atherectomy system that is inserted into target material may be referred to as a “proximal end,” and an opposite end of the atherectomy system may be referred to as a “distal end.” In some embodiments, the rotation of the helical screw further advances cut portions of the target material through the tubular element toward the distal end of the atherectomy system.

In some embodiments, at block 2018, the atherectomy process 2000 includes removing cut target material from the tubular structure. For example, cut target material may be further suctioned and/or pushed through the tubular element 105 of the atherectomy system 100 and aspirated out of the tubular structure. Additionally, or alternatively, in some embodiments, the cut target material may be flushed out of the tubular structure. In some embodiments, the cut target material is received into a collection catheter, which may be removed from the subject to extract the target material. The extraction of the cut target material may occur simultaneous or asynchronously to further advancing and rotation of the helical screw and one or more wires within the tubular structure.

### Conclusion

While some embodiments described herein relate to atherectomy systems, one of ordinary skill in the art will appreciate that the teachings herein may also apply to a wide range of medical procedures and apparatuses. The embodiments described herein may also be scalable to accommodate at least the aforementioned applications. Various components of embodiments described herein can be added, removed, reorganized, modified, duplicated, or the like as one skilled in the art would find convenient and/or necessary to implement a particular application in conjunction with the teachings of the present disclosure. In some embodiments, specialized features, characteristics, materials, components, and/or equipment may be applied in conjunction with the teachings of the present disclosure as one skilled in the art would find convenient and/or necessary to implement a particular application.

Moreover, many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of any appended claims. Moreover, although the foregoing descriptions and the associated drawings describe example embodiments in the context of certain example combinations of elements and/or functions, it should be appreciated that different combinations of elements and/or functions can be provided by alternative embodiments without departing from the scope of any appended claims. In this regard, for example, different combinations of elements and/or functions than those explicitly described above are also contemplated as can be set forth in some of any appended claims. Although specific terms are employed herein, they are used in a generic and descriptive sense only and not for purposes of limitation.

That which is claimed is:

1. A system, comprising:
at least one wire comprising opposed ends, the opposed ends defining a length of the at least one wire;
a helical screw coupled to the at least one wire along the length of the at least one wire between the opposed ends, wherein the helical screw is configured to rotate with the at least one wire; and
a tubular element threaded over a portion of the length, wherein:
the tubular element comprises a tip comprising at least one blade structure;
upon rotation of the at least one wire, the helical screw is configured to generate a negative pressure within the tubular element; and
the negative pressure within the tubular element is configured to draw material toward the tip to enable cutting of the material against the helical screw and the at least one blade structure.

2. The system of claim 1, wherein a respective blade structure of the at least one blade structure comprises:
a rounded external surface; and
at least one sharpened interior edge.

3. The system of claim 2, wherein the at least one blade structure comprises a plurality of blade structures in a radial arrangement.

4. The system of claim 3, wherein the respective rounded external surfaces of the plurality of blade structures define a hemisphere comprising a central void and a plurality of radially spaced voids between respective blade structures.

5. The system of claim 1, wherein the tubular element is configured to remain static relative to the rotation of the at least one wire.

6. The system of claim 1, further comprising a termination cap secured over the at least one wire at one of the opposed ends.

7. The system of claim 6, wherein a connection between the termination cap and the at least one wire comprises at least one polymer material.

8. The system of claim 6, wherein a connection between the termination cap and the at least one wire comprises at least one solder material.

9. The system of claim 1, further comprising:
at least one rotation mechanism operatively connected the at least one wire, the at least one rotation mechanism configured to rotate the at least one wire upon activation.

10. The system of claim 1, wherein the at least one wire comprises a plurality of wires.

11. The system of claim 1, wherein the at least one wire comprises stainless steel.

12. The system of claim 1, wherein the at least one wire comprises nitinol.

13. The system of claim 1, wherein the at least one wire comprises at least one radiopaque material.

14. The system of claim 1, wherein the helical screw comprises stainless steel.

15. The system of claim 1, wherein the helical screw comprises tungsten carbide.

16. The system of claim 1, wherein the helical screw comprises at least one radiopaque material.

17. A kit comprising:
at least one system, comprising:
at least one wire comprising opposed ends, the opposed ends defining a length of the at least one wire;
a helical screw coupled to the at least one wire along the length of the at least one wire between the opposed ends, wherein the helical screw is configured to rotate with the at least one wire; and
a tubular element threaded over a portion of the length, wherein:
the tubular element comprises a tip comprising at least one blade structure;
upon rotation of the at least one wire, the helical screw is configured to generate a negative pressure within the tubular element; and
the negative pressure within the tubular element is configured to draw material toward the tip to enable cutting of the material against the helical screw and the at least one blade structure; and
at least one guidewire, wherein the at least one system is configured to be deployed to a target site via the at least one guidewire.

18. The kit of claim 17, wherein the at least one system comprises:
a first system comprising a respective tubular element of a first bore size; and
a second system comprising a respective tubular element of a second bore size, wherein the second bore size exceeds the first bore size.

19. A method comprising:
deploying a guidewire to a tubular structure of a subject, the tubular structure comprising a target material;
inserting a system into the tubular structure via the guidewire, the system comprising:
at least one wire comprising opposed ends, the opposed ends defining a length of the at least one wire;
a helical screw coupled to the at least one wire along the length of the at least one wire between the opposed ends, wherein the helical screw is configured to rotate with the at least one wire; and
a tubular element threaded over a portion of the length, wherein:
the tubular element comprises a tip comprising at least one blade structure;
rotating the at least one wire via a rotation mechanism coupled to one of the opposed ends of the at least one wire; and
advancing the tip of the helical screw into the target material, wherein:
the rotating of the at least one wire causes rotation of the helical screw;
the rotation of the helical screw generates a negative pressure within the tubular element to draw the target material to the tip of the tubular element; and
the at least one blade structure and the helical screw cut the target material in a region proximate to the tip of the tubular element.

20. The method of claim 19, wherein:
the cutting of the target material is caused by collective engagement of the target material with the helical screw and the at least one blade structure.
